(19) United States
(12) Patent Application Publication
(10) Pub. No.: US 2005/0096294 A1
(43) Pub. Date: May 5, 2005

(54) Novel anti-coagulant

(76) Inventors: Takashi Komai, Mie (JP); Keiichi Miyamoto, Mie (JP); Mototake Tsutsui, Kanagawa (JP); Ikuo Sato, Kanagawa (JP); Shinichi Takasaki, Kanagawa (JP)

Correspondence Address: WENDEROTH, LIND & PONACK, L.L.P., 2033 K STREET N. W., SUITE 800, WASHINGTON, DC, 20006-1021, US

(21) Appl. No.: 10/697,878

(22) Filed: Oct. 31, 2003

(51) Int. Cl. A61K 31/737; C07H 13/12

(52) U.S. Cl. 514/54; 536/118

(57) ABSTRACT

Provided by the present invention are an anti-coagulant comprising a polysaccharide having a structural unit in which an abundance ratio of glucose, glucuronic acid and rhamnose is 2:1:1 mole, preferably a polysaccharide having a structural unit represented by Formula (1) and more preferably a compound obtained by partially sulfating a hydroxyl group of gellan and an anti-thrombus agent or a blood contact face-treating agent for medical equipment comprising this anti-coagulant.

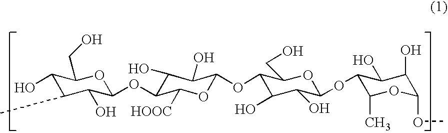

### FIELD OF THE INVENTION

The present invention relates to an anti-coagulant used for preventing or treating symptoms of thrombosis by inhibiting coagulation of blood or used for surface treatment of medical equipment.

### BACKGROUND OF THE INVENTION

Heparin is present in an intestine or a lung of mammals (bovine, lambs and pigs). Heparin is mucopolysaccharide having a sulfate group and has an anti-coagulant action. Accordingly, heparin is used for treatment and prevention of diseases caused by abnormal extracorporeal circulating blood pathway and anti-coagulation of extracorporeal circulating blood in hemodialysis or when using an artificial heart-lung device. Further, heparin is used in order to prevent medical equipment introduced into an organism from coagulating blood.

Coagulation of blood is a complicated system (cascade process) in which a large number of proteolytic enzymes are activated each other in a fixed order. In this cascade process, a mechanism by which heparin acts becomes gradually apparent.

Thus, heparin is a valuable medicine. However, when a very large amount of heparin is administered, complications such as hemorrhage are observed in a certain case. This is because heparin acts on both of an intrinsic pathway and an extrinsic pathway among blood coagulation pathways.

In recent years, it has become clear that a low molecular weight fraction (LMW heparin) of heparin acts only on an intrinsic pathway blood coagulation factor unlike heparin. This means that LMW heparin has a specific anti-Xa factor activity analogous to that of heparin and that it has a low activity to inhibit whole coagulation. That is, LMW heparin has an anti-coagulation activity as is the case with heparin, but it is less likely to cause complications such as hemorrhage.

The following production processes for LMW heparin have been investigated:
Chemical degradation in which heparin is decomposed by acid and alkali and turned into a low molecular weight (for example, Japanese Patent Application Laid-Open No. 191801/1988 and Japanese Patent Application Laid-Open No. 64102/1990).
Enzymatic degradation in which heparin is depolymerized by an enzyme and turned into a low molecular weight (for example, Japanese Patent Application Laid-Open No. 247297/1991).

Thus, LMW heparin is produced using heparin as a raw material. Heparin is expensive and requires such production steps, and therefore LMW heparin becomes very expensive. Further, as described above, heparin is extracted from an intestine and a lung of bovine, lambs and pigs, and therefore it is very difficult to prevent virus and prion protein from being mixed into heparin. Accordingly, heparin extracted from cattle organs has been inhibited from being used since the prevalence of bovine spongiform encephalopathy (BSE). This has been accompanied with a sudden rise in the price of heparin. A safe, inexpensive and novel anti-coagulant is required because of such reasons.

### SUMMARY OF THE INVENTION

The anti-coagulant of the present invention is a polysaccharide obtained by using a raw material of a polysaccharide having a structural unit in which an abundance ratio of glucose, glucuronic acid and rhamnose is 2:1:1 mole to partially sulfate a hydroxyl group of the above raw material polysaccharide, or a compound having the sulfated polysaccharide as a partial structure.

### DETAILED DESCRIPTIONS

Intensive researches repeated by the present inventors in order to solve the problems described above have resulted in finding that a polysaccharide obtained by using a raw material of a polysaccharide having a structural unit in which an abundance ratio of glucose, glucuronic acid and rhamnose is 2:1:1 mole to partially sulfate a hydroxyl group of the above raw material polysaccharide has the same blood anti-coagulation activity as those of heparin and LMW heparin.

The present invention comprises the following structures.
(1) An anti-coagulant comprising a polysaccharide obtained by using a raw material of a polysaccharide having a structural unit in which an abundance ratio of glucose, glucuronic acid and rhamnose is 2:1:1 mole to sulfate 8 to 80% of a hydroxyl group contained in the above raw material polysaccharide or a compound having the sulfated polysaccharide as a partial structure.
(2) The anti-coagulant as described in the above item (1), wherein the raw material polysaccharide is a polysaccharide having a structural unit represented by the following Formula (1): 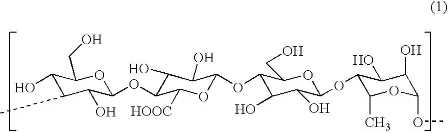
(3) The anti-coagulant as described in the above item (1), wherein the raw material polysaccharide is gellan.
(4) The anti-coagulant as described in the above item (1), comprising the polysaccharide obtained by sulfating 20 to 50% of a hydroxyl group contained in the raw material polysaccharide or the compound having the sulfated polysaccharide as a partial structure.
(5) The anticoagulant as described in the above item (1), wherein the sulfated polysaccharide has a mean molecular weight of 1 to 1000 KDa.
(6) The anti-coagulant as described in the above item (1), wherein the sulfated polysaccharide has a mean molecular weight of 1 to 30 KDa.
(7) An anti-thrombus agent comprising the anti-coagulant as described in any of the above items (1) to (6).
(8) The anti-thrombus agent as described in the above item (7), capable of being used for prevention and treatment of myocardial infarction, cerebral infarction or venous thrombosis.
(9) The anti-thrombus agent as described in the above item (7) or (8), obtained by processing the anti-coagulant into the form of a unit preparation for intravenous administration, intestinal administration or oral administration.
(10) A blood contact face-treating agent for medical equipment, comprising the anti-coagulant as described in any of the above items (1) to (6).
(11) Medical equipment treated using the blood contact face-treating agent as described in the above item (10).
(12) A catheter, an injector for collecting blood, an artificial organ, an infusion pack or an infusion tube treated using the blood contact face-treating agent as described in the above item (10).

The present invention shall be explained below in details.

The first present invention is an anti-coagulant comprising a polysaccharide obtained by partially sulfating a hydroxyl group of a raw material polysaccharide having a structural unit in which an abundance ratio of glucose, glucuronic acid and rhamnose is 2:1:1 mole. A degree of substitution for sulfonation of a hydroxyl group, that is, a proportion in which a hydroxyl group contained in the polysaccharide described above before used as a raw material is sulfated is 8 to 80%, preferably 20 to 50%. The sulfated polysaccharide has a mean molecular weight of preferably 1 to 1000 KDa, more preferably 1 to 30 KDa.

The polysaccharide used as a raw material for the sulfated polysaccharide constituting the anti-coagulant of the present invention may be a chemically synthesized product, a nature-originating product obtained by fermentation with microorganisms or an alga extract. The origin of the polysaccharide of the raw material shall not specifically be restricted. Also, the polysaccharide of the raw material can be turned into a low molecular weight and then subjected to sulfonation reaction. A method for turning into a low molecular weight includes hydrolysis by hydrochloric acid, sulfuric acid, trifluoroacetic acid, other acids and alkaline compounds such as sodium hydroxide and a decomposition method by an enzyme. The method for turning into a low molecular weight includes the other methods and shall not specifically be restricted.

A polysaccharide having a structural unit represented by the following Formula (1) can be given as the specific example of the polysaccharide of the raw material:

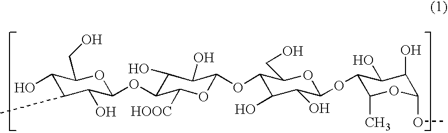

To be more specific, the polysaccharide of the raw material includes the polysaccharide comprising the structural unit represented by Formula (1), that is, gellan (CAS 71010-52-1) obtained by deacylating a polysaccharide produced by Pseudomonas elodea. Gellan is a polysaccharide comprising glucose, glucuronic acid and rhamnose as principal components and can be obtained at a low cost in a large amount. Accordingly, it can preferably be used in the present invention.

A usually known method can be applied to a method for sulfating the polysaccharide. It includes, for example, a method in which chlorosulfonic acid is acted in dimethylforamide (DMF), introduced by K. Miyamoto et al. to International Journal of Biological Macromolecules, 28, 381 (2001) and a method in which a DMF/SO₃ complex is acted in DMF. Further, in addition thereto, a method in which a sulfuric anhydride complex such as a dioxane-SO₃ complex, a trimethylamine-SO₃ complex and a pyridine-SO₃ complex is acted can be used as the reacting method. The sulfated polysaccharide having an optional molecular weight and degree of substitution for sulfation can be obtained by changing a molecular weight of the polysaccharide of the raw material and the reaction conditions.

The sulfated polysaccharide described above is preferably used for the anti-coagulant of the present invention. Further, the compound having the sulfated polysaccharide described above as a partial structure can be used as well for the anti-coagulant of the present invention. The examples of the compound into which the sulfated polysaccharide is introduced include a polysaccharide having an amino group, a polyamino acid having an amino group and a polysaccharide into which an amino group is introduced. The specific example of the polysaccharide having an amino group is chitosan. The specific example of the polyamino acid having an amino group is poly-L-lysine. The specific example of the polysaccharide into which an amino group is introduced is aminated cellulose. In addition thereto, any compound may be combined with the sulfated polysaccharide as long as the blood anti-coagulating property of the sulfated polysaccharide of the present invention is not lost.

The examples of a method for introducing the sulfated polysaccharide into the compound described above include the following methods. They are a method in which a carboxyl group of the sulfated polysaccharide is combined with an amino group of the compound by using water-soluble carbodiimide as a catalyst and a method in which a reducing end aldehyde group of the sulfated polysaccharide is reacted with an amino group of the compound under a weak alkaline condition and bonded thereto by treating with a reducing agent (sodium tetrahydroborate, dimethylamineborane and the like). In addition thereto, the bonding method shall not specifically be restricted as long as the anti-coagulating property of the sulfated polysaccharide of the present invention is not lost.

The second present invention is an anti-thrombus agent comprising the novel anti-coagulant of the first present invention.

The anti-coagulant prepared by the process described above can be converted into a form which can be administered as a medicine by processes usually used for heparin and LMW heparin. For example, the anti-coagulant of the present invention can be used by dissolving in water in order to prepare an injection preparation. In this case, adjuvants (hojyo-zai) (a preservative, a kind of a salt and the like) which are allowed to be used for preparations can be added to the injection preparation. Such injection preparation is clinically used in the form of hypodermic or intravenous injection (to be suitable, intermittently) or infusion. Pulmonary administration by spray inhalation and percutaneous administration by an ointment and a cream and mucosal administration by a suppository can also be used as the other administrating methods.

The anti-thrombus agent prepared can be used for prevention and treatment of myocardial infarction, cerebral infarction or venous thrombosis. These diseases are triggered by thrombus formed in a blood vessel by blood coagulation. The present anti-thrombus agent can retard a progress in thrombus formation at an initial stage of thrombus formation, and therefore it is very effective for the purpose of prevention and treatment. These administering methods of the anti-thrombus agent have to be suitably carried out according to the morbid state. The principal administering method of the present anti-thrombus agent includes hypodermic administration and intravenous administration by injection, oral administration by tableting and intestinal administration in the form of a suppository. In addition thereto, it is no problem to use any administering methods and drug forms as long as the suitable forms such as percutaneous administration by an ointment and a wet pack and pulmonary administration by spray inhalation are selected.

The third present invention is a blood contact face-treating agent for medical equipment, comprising the novel anti-coagulant of the first present invention. Further, it is medical equipment treated using the above blood contact face-treating agent.

Medical equipment represented by a catheter, an injector for collecting blood, an artificial organ, an infusion pack and an infusion tube which are brought into contact with blood are subjected to surface treatment by heparin for the purpose of preventing blood coagulation. The anti-coagulant of the present invention can be used as well for preventing blood coagulation on the surface of medical equipment as is the case with heparin. The example of the specific method for treating the surface of medical equipment includes a method in which the surface of medical equipment is modified with a substituent which can physically adsorb the sulfated polysaccharide or can be chemically combined with it to physically adsorb or chemically combine the sulfated polysaccharide onto the surface of medical equipment. Further, it includes a method in which the sulfated polysaccharide is combined with the other compound having a substituent to combine the sulfated polysaccharide onto the surface of medical equipment making use of the compound. Also, it includes a method in which a part of the sulfated polysaccharide is substituted with the other substituent to combine the sulfated polysaccharide onto the surface of medical equipment. In addition thereto, the coagulant of the present invention may be combined onto the surface of medical equipment by any method as long as the anti-coagulating property is not lost.

The present invention has made it possible to provide the novel anti-coagulant having the same anti-coagulating action as that of heparin and the same specificity as that of LMW heparin at a lower cost than those of heparin and LMW heparin. This anti-coagulant can be used for an anti-thrombus agent and a blood contact face-treating agent for medical equipment.

### EXAMPLES

The present invention shall be explained below in details with reference to examples and comparative examples, but the present invention shall not be restricted by these examples. Definitions of terms used in the examples and the measuring methods shall be explained below.

(1) Mean Molecular Weight (KDa):

The synthesized sulfated polysaccharide was dissolved in a 0.2 mol/1-NaCl aqueous solution (adjusted with ion-exchanged water) in a concentration of 1.0 mg/ml to measure the mean molecular weight by gel filtration according to high performance liquid chromatography (HPLC) using the same NaCl aqueous solution as an eluate. Shodex Ionpak KS-804 and KS-G were used for the columns. The eluted matters were detected by means of a refractive index detector. A working curve of eluting time and a molecular weight was prepared from pullulan (Shodex STANDARD P-82) having a known molecular weight measured separately, and a mean molecular weight of the substance concerned was determined by applying to the working curve.

(2) Degree of Substitution for Sulfation (%) of Hydroxyl group:

A sulfated proportion of hydroxyl groups contained in the raw material polysaccharide was shown by percentage. The total S content of the synthesized sulfated polysaccharide was measured by means of ICP, and an amount of S liberated from the sulfated polysaccharide was measured by means of ion chromatography. The degree of substitution for sulfation of a hydroxyl group was calculated from a binded S amount obtained by deducting the liberated S amount from the total S content.

(3) Normal Blood Coagulation Time (Second):

The sulfated polysaccharide 50 μl was separately put (the concentration was adjusted so that the final concentration was a measured concentration) in advance into plastic test tubes (3.3 ml blood collected tube, manufactured by IWAKI Co., Ltd.), and each 1 ml of blood immediately after collected from a normal person was put into the test tubes containing the sulfated polysaccharide and quickly mixed, and the test tubes were repetitively inclined to measure time (second) in which the fluidity disappeared.

(4) Activated Partial Thromboplastin Time (APTT):

The present method is a method for inspecting dynamics of intrinsic pathway coagulation. The present method has sensitivity to qualitative and quantitative abnormality in the factor XII, the factor XI, high molecular weight kininogen, prekallikrein, the factor IX, the factor VIII, the factor X, the factor V, the factor II (prothrombin) and the factor I (fibrinogen). First, normal (normal volunteer) blood citrate-collected (collected in a plastic test tube charged with 3.13 wt % sodium citrate of 1/10 volume) was subjected to centrifugal separation at 3000 rpm for 10 minutes to obtain supernatant plasma. The APTT time was obtained by mixing plasma for inspection with an APTT reagent and then measuring fibrin-depositing time (second) after adding a calcium chloride solution (using an automatic measuring apparatus). The sulfated polysaccharide was added to the plasma for inspection in an addition concentration of 0.001 to 1 mg/ml, and normal plasma was used for a standard control.

(5) Prothrombin Time (PT):

The present method is a method for inspecting dynamics of extrinsic pathway coagulation. The present method has sensitivity to qualitative and quantitative abnormality in the factor II (prothrombin), the factor V, the factor VII, the factor X and the factor I (fibrinogen), and it is scarcely influenced by the factor 1× and the factor VIII. First, normal (normal volunteer) blood citrate-collected (collected in a plastic test tube charged with 3.13 wt % sodium citrate of 1/10 volume) was subjected to centrifugal separation at 3000 rpm for 10 minutes to obtain supernatant plasma. The PT time was obtained by mixing plasma for inspection with a PT reagent (a mixed solution of tissue thromboplastin and calcium chloride) to measure fibrin-depositing time (second) (using an automatic measuring apparatus). The sample of the sulfated polysaccharide was added to the plasma for inspection in an addition concentration of 0.001 to 1 mg/ml, and normal plasma was used for a standard control.

### Example 1

Gellan (manufactured by Wako Pure Chemical Industries, Ltd.) 2 g was added in advance to a 0.5 mol/liter-trifluoroacetic acid aqueous solution 200 ml, and they were hydrolyzed at 80° C. for 30 minutes. Low molecular weight gellan thus obtained was added to DMF 5 g under nitrogen gas sealing and swollen by stirring at a room temperature for 10 hours. Then, the temperature of the reaction solution was elevated to 40° C., and 14 g of a DMF/SO₃ complex (SO₃: 18 wt %) was added thereto to carry out reaction for 6 hours. After completing the reaction, the reaction solution was cooled on ice, and 0.3 g of water was added to decompose the unreacted DMF/SO₃ complex to terminate the reaction. Subsequently, a two times volume of ethanol was added to the reaction solution to precipitate the reaction product, and it was recovered by filtering. The precipitate thus recovered was dissolved in 20 ml of ion-exchanged water, and the solution was neutralized by 1 mol/1-NaOH and precipitated again by adding a two times volume of ethanol to recover the precipitate. Thereafter, refining and washing were repeated three times in total by the present method, and the precipitate thus obtained was dried at 50° C. under reduced pressure for a day to obtain 1.7 g (yield: 61%) of a powder of gellan sulfate. A mean molecular weight of gellan sulfate thus obtained was measured by the method described above to find that it was 8.4 KDa. Further, a degree of substitution for sulfonation of a hydroxyl group was 24.4%.

APTT time and PT time of gellan sulfate thus obtained were measured. As a result thereof, the APTT time of a sample to which gellan sulfate was not added was 30 seconds, but that of a sample to which gellan sulfate was added in a concentration of 1 mg/ml was extended to 95 seconds. In the case of the PT time, significant extension of the time was not observed.

### Example 2

Sulfation was carried out by the method according to Example 1, except that normal gellan 2 g which was not turned into a low molecular weight was used as the raw material and chlorosulfonic acid 3.6 g was used for a sulfating agent and that the reaction was carried out at a temperature of 50 C. As a result thereof, resulting gellan sulfate had a mean molecular weight of 23 KDa, and the hydroxyl group thereof had a degree of substitution for sulfonation of 36.6%. An APTT time and a PT time of gellan sulfate obtained and the coagulation time obtained using normal blood were measured to obtain the following results shown in Table 1.

TABLE 1

| Addition | | | |
|---|---|---|---|
| concentration | | | Normal blood |
| of gellan sulfate | PT | APTT | coagulation time |
| (mg/ml) | (second) | (second) | (minute) |
| 0 | 12.6 | 35 | 42.5 |
| (standard control) | | | |
| 0.001 | 11.6 | 38.1 | 54 |
| 0.01 | 11.7 | 30 | 93 |
| 0.1 | 15.8 | 300 | 230 |
| | | or more | |
| 1 | 300 | 300 | 600 |
| | or more | or more | or more |

### Example 3

Sulfation was carried out on the same conditions as in Example 2, except that gellan which was turned into a low molecular weight by the method according to Example 1 was used as the raw material. As a result thereof, gellan sulfate having a mean molecular weight of 13 KDa and a degree of substitution for sulfation of a hydroxyl group of 39.8% was obtained. An APTT time and a PT time of gellan sulfate obtained and the coagulation time obtained using normal blood were measured to obtain the following results shown in Table 2.

TABLE 2

| Addition | | | |
|---|---|---|---|
| concentration | | | Normal blood |
| of gellan sulfate | PT | APTT | coagulation time |
| (mg/ml) | (second) | (second) | (minute) |
| 0 | 12.6 | 35 | 42.5 |
| (standard control) | | | |
| 0.001 | 11.3 | 49 | 58.9 |
| 0.01 | 11.8 | 103.5 | 138.8 |
| 0.1 | 19.3 | 300 | 216.3 |
| | | or more | |
| 1 | 300 | 300 | 600 |
| | or more | or more | or more |

### Example 4

Sulfation was carried out on the same conditions as in Example 3, except that an addition amount of chlorosulfonic acid was changed to 18 g to obtain gellan sulfate having a molecular weight of 9 KDa and a degree of substitution for sulfation of a hydroxyl group of 46.4%. An APTT time and a PT time of gellan sulfate obtained and the coagulation time obtained using normal blood were measured to obtain the following results shown in Table 3.

TABLE 3

| Addition | | | |
|---|---|---|---|
| concentration | | | Normal blood |
| of gellan sulfate | PT | APTT | coagulation time |
| (mg/ml) | (second) | (second) | (minute) |
| 0 | 12.6 | 35 | 42.5 |
| (standard control) | | | |
| 0.001 | 13 | 45 | 39.9 |
| 0.01 | 12 | 45 | 72 |
| 0.1 | 14 | 300 | 600 |
| | | or more | or more |
| 1 | 300 | 300 | 600 |
| | or more | or more | or more |

### COMPARATIVE EXAMPLE 1

Sulfation was carried out by the method according to Example 1, except that an addition amount of the DMF/SO₃ complex was changed to 0.4 g. As a result thereof, gellan sulfate having a mean molecular weight of 9 KDa and a degree of substitution for sulfation of a hydroxyl group of 5% was obtained. The gellan sulfate 1 mg/ml was added, but a coagulation time of normal blood was not observed to be extended.

### COMPARATIVE EXAMPLE 2

An APTT time and a PT time of heparin (manufactured by Scientific Protein Laboratories) in place of gellan sulfate and the coagulation time obtained using normal blood were measured to obtain the following results shown in Table 4.

TABLE 4

| Addition | | | |
|---|---|---|---|
| concentration | | | Normal blood |
| of heparin | PT | APTT | coagulation time |
| (mg/ml) | (second) | (second) | (minute) |
| 0 | 12.6 | 35 | 42.5 |
| (standard control) | | | |
| 0.001 | 13 | 50 | 105.6 |
| 0.01 | 13 | 300 | 600 |
| | | or more | or more |
| 0.1 | 300 | 300 | 600 |
| | or more | or more | or more |
| 1 | 300 | 300 | 600 |
| | or more | or more | or more |

### COMPARATIVE EXAMPLE 3

Measured was a blood coagulation time of typical commercial chondroitin sulfate (manufactured by Wako Pure Chemical Industries, Ltd.) as sulfated polysaccharide in place of gellan sulfate using normal blood. In this case, a coagulation time of normal blood was not observed at all to be extended even by addition of 0.01 mg/ml.

1. An anti-coagulant comprising a polysaccharide obtained by using a raw material of a polysaccharide having a structural unit in which an abundance ratio of glucose, glucuronic acid and rhamnose is 2:1:1 mole to sulfate 8 to 80% of a hydroxyl group contained in the above raw material polysaccharide or a compound having the sulfated polysaccharide as a partial structure.

2. The anti-coagulant as described in claim 1, wherein the raw material polysaccharide is a polysaccharide having a structural unit represented by the following Formula (1):

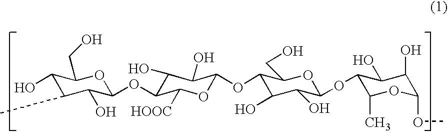

3. The anti-coagulant as described in claim 1, wherein the raw material polysaccharide is gellan.

4. The anti-coagulant as described in claim 1, comprising the polysaccharide obtained by sulfating 20 to 50% of a hydroxyl group contained in the raw material polysaccharide or the compound having the sulfated polysaccharide as a partial structure.

5. The anti-coagulant as described in claim 1, wherein the sulfated polysaccharide has a mean molecular weight of 1 to 1000 KDa.

6. The anti-coagulant as described in claim 1, wherein the sulfated polysaccharide has a mean molecular weight of 1 to 30 KDa.

7. An anti-thrombus agent comprising the anti-coagulant as described in any of claims 1 to 6.

8. The anti-thrombus agent as described in claim 7, capable of being used for prevention and treatment of myocardial infarction, cerebral infarction or venous thrombosis.

9. The anti-thrombus agent as described in claim 7 or 8, obtained by processing the anti-coagulant into the form of a unit preparation for intravenous administration, intestinal administration or oral administration.

10. A blood contact face-treating agent for medical equipment, comprising the anti-coagulant as described in any of claims 1 to 6.

11. Medical equipment treated using the blood contact face-treating agent as described in claim 10.

12. A catheter, an injector for collecting blood, an artificial organ, an infusion pack or an infusion tube treated using the blood contact face-treating agent as described in claim 10.
